SEQUENCE LISTING (6 sequences)

SEQ ID NO: 1 - protein, 57 aa, Homo sapiens
AQEPVKGPVSTKPGSCPIILIRCAMLNPPNRCLKDTDCPGIKKCCEGSCGMACFVPQ

SEQ ID NO: 2 - protein, 117 aa, Homo sapiens
MRASSFLIVVVFLIAGTLVLEAAVTGVPVKGQDTVKGRVPFNGQDPVKGQVSVKGQDKVKAQEPVKGPVSTKPGSCPIILIRCAMLNPPNRCLKDTDCPGIKKCCEGSCGMACFVPQ

SEQ ID NO: 3 - DNA, 623 bp, Homo sapiens
  source 1..623: mol_type = other DNA
aggccaagctggactgcataaagattggtatggccttagctcttagccaaacaccttcctgacaccatgagggccagcagcttcttgatcgtggtggtgttcctcatcgctgggacgctggttctagaggcagctgtcacgggagttcctgttaaaggtcaagacactgtcaaaggccgtgttccattcaatggacaagatcccgttaaaggacaagtttcagttaaaggtcaagataaagtcaaagcgcaagagccagtcaaaggtccagtctccactaagcctggctcctgccccattatcttgatccggtgcgccatgttgaatccccctaaccgctgcttgaaagatactgactgcccaggaatcaagaagtgctgtgaaggctcttgcgggatggcctgtttcgttccccagtgagagggagccggtccttgctgcacctgtgccgtccccagagctacaggccccatctggtcctaagtccctgctgcccttccccttcccacactgtccattcttcctcccattcaggatgcccacggctggagctgcctctctcatccactttccaataaagagttccttctgctccaaaaaaaaaaaaaaaaaaaaaaaaaaaa

SEQ ID NO: 4 - protein, 6 aa, synthetic construct
  REGION 1..6: note = Repeat sequence
  VARIANT 4: note = X can be any naturally occurring amino acid
GQDXVK

SEQ ID NO: 5 - protein, 38 aa, Homo sapiens
CPIILIRCAMLNPPNRCLKDTDCPGIKKCCEGSCGMAC

SEQ ID NO: 6 - protein, 4 aa, synthetic construct
  REGION 1..4: note = AAPVpNA synthetic peptide
  MOD_RES 1: note = methoxy-succinyl alanine
  MOD_RES 4: note = N-(4-Nitrophenyl)-L-valinamide
AAPV

(19) United States
(12) Patent Application Publication
(10) Pub. No.: US 2023/0285523 A1
(43) Pub. Date: Sep. 14, 2023

(54) USE OF ELAFIN IN THE TREATMENT OF COVID-19

(71) Applicant: tiakis Biotech AG, Kiel (DE)

(72) Inventor: Oliver WIEDOW, Kiel (DE)

(21) Appl. No.: 18/068,946

(22) Filed: Dec. 20, 2022

Related U.S. Application Data
(60) Provisional application No. 63/353,705, filed on Jun. 20, 2022.
(60) Provisional application No. 63/291,740, filed on Dec. 20, 2021.

(51) Int. Cl. A61K 38/57; A61P 31/14

(52) U.S. Cl. CPC A61K 38/57; A61P 31/14

(57) ABSTRACT

The present invention relates to the use of elafin or a homologue, fragment or derivative thereof in the treatment of COVID-19, in particular any disorder or disease associated with an infection with the SARS-CoV-2 virus. The present invention also relates to a respective method of treatment for COVID-19 and composition comprising elafin or a homologue, fragment or derivative thereof for use in said treatment. The present invention additionally or concomitantly relates to the use of elafin or a homologue, fragment or derivative thereof for the treatment and/or prevention of complications associated with COVID-19 and/or progression to severe disease upon infection with SARS-CoV-2 virus.

### FIELD OF THE INVENTION

The present disclosure relates to the use of elafin in the treatment of COVID-19, in particular for the treatment of any disorder or disease associated with an infection with the SARS-CoV-2 virus. The present disclosure also relates to a method of treatment for COVID-19 using elafin, in particular for the treatment of any disorder or disease associated with an infection with the SARS-CoV-2 virus. The present disclosure further relates to a composition comprising elafin for use in the treatment of COVID-19, in particular for the treatment of any disorder or disease associated with an infection with the SARS-CoV-2 virus. The present disclosure additionally or concomitantly relates to the use of elafin for the treatment and/or prevention of complications associated with COVID-19 and/or for the prevention of progression to severe disease upon infection with SARS-CoV-2 virus. Furthermore, the present disclosure relates, in one particular embodiment, to the use of elafin to alleviate respiratory distress in a subject infected with the SARS-CoV-2 virus. Additionally, the present disclosure relates, in another particular embodiment, to the use of elafin to facilitate and accelerate the removal of the need for oxygen supply to a subject infected with the SARS-CoV-2 virus.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: PRBI_002_02WO_SeqList_ST26.xml, date of creation: Nov. 16, 2022, file size: 8,921 bytes).

### BACKGROUND OF THE INVENTION

Elafin is a recombinant human protein, known to act as a reversible tight binding inhibitor of elastase and the closely related serine protease proteinase 3. Elafin can be taken up from the extracellular medium by monocytes (Butler et al.) and vascular endothelial cells (Nickel et al. 2013).

Elafin can interfere with intracellular signaling events. In the monocytic cell line U937 elafin inhibited the LPS-induced activation of the transcription factors AP-1 and NF-κB via an effect on the ubiquitin-proteasome pathway, with no inhibition of peptidase activities associated with the 20 S proteasome. This led to inhibition of the production of the potent proinflammatory factor macrophage chemokine MCP-1 (macrophage chemoattractant protein-1; CCL2) (Butler et al.).

Over-expression of elafin by gene transfer suppressed an inflammatory response in cultured human umbilical vein endothelial cells (HUVEC). The release of IL-8 by HUVEC in response to a challenge with oxidized low-density lipoprotein, LPS and TNFα was reduced by elafin overexpression. In addition, elafin overexpression in macrophages attenuated the LPS-stimulated release of the proinflammatory cytokine TNFα. In both cell types these effects were associated with reduced activation of the inflammatory transcription factor NF-κB, through up-regulation of IκBα (Henriksen et al.).

Despite the plethora of scientific research on elafin, the one commonly known and established effect of elafin is its effect on elastase (and the serine protease proteinase 3). Elafin has been reported to be a highly specific, potent and reversible inhibitor for neutrophil-derived elastase and proteinase 3. A further effect of elafin is its effect on the release of troponins, in particular cardiac troponin I, cardiac troponin T or skeletal troponin T.

Accordingly, elafin has been proposed as a suitable agent for the treatment or prevention of diseases shown to be the result of abundant amounts of elastase, for example in SIRS (systemic inflammation response syndrome) or MODS (multiple-organ dysfunction syndrome). Elafin inhibits the formation of neutrophil exosomes and neutrophil extracellular traps, inhibits NFkB and restores pathologic bone morphogenic protein signaling. In vivo, elafin reduces vascular smooth muscle cell proliferation and vascular occlusion, and promotes alveolar formation.

Elafin has been subjected to numerous preclinical investigations in animals in a variety of disease models. It suppresses reperfusion injury occurring after heart transplantation, myocardial infarction, and skeletal muscle ischemia. It is also able to revert the pathology in pulmonary arterial hypertension and to reduce ventilator induced injury. Furthermore, transgenic animals expressing human elafin in the cardiovascular system became much more resistant to viral myocarditis, ventilator injury, vascular injury, angioplasty, vein graft degeneration as well as pulmonary arterial hypertension induced by hypoxia.

The COVID-19 pandemic has exploded since cases were first reported in China in December 2019. As of November, 2022, more than 630 million confirmed cases of COVID caused by severe acute respiratory syndrome coronavirus 2 (SARS-CoV-2)—have been reported globally, including more than 6.5 million deaths (https://covid19.who.int/).

Individuals of all ages are at risk for infection and severe disease. However, the probability of severe and critical COVID-19 disease is higher in unvaccinated people aged ≥60 years, those living in a nursing home or long-term care facility, and those with chronic medical conditions. Among those with available data on health conditions, 32% had cardiovascular disease, 30% had diabetes, and 18% had chronic lung disease. Other conditions that may lead to a high risk for severe COVID-19 include cancer, kidney disease, obesity, sickle cell disease, and immunocompromi sing conditions (htps://www.cdc.gov/coronavirus/2019-ncov/cases-updates/cases-in-us.html).

Two main processes are thought to drive the pathogenesis of COVID-19. Early in the course of the infection, the disease is primarily driven by replication of SARS-CoV-2. Later in the course of infection, the disease is driven by an exaggerated immune/inflammatory response to the virus leading predominantly to pulmonary tissue damage, in most severe cases to multiple organ failure and death.

The US National Institutes of Health has, in October 2022, issued updated COVID-19 Treatment Guidelines (https://www.covid19treatmentguidelines.nih.gov/). However, the overall effectiveness of current therapies in preventing and/or ameliorating disease progression in severe or critical patients is still low. There exists an urgent need for further, generally applicable, approaches for the treatment of COVID-19, especially for the treatment and/or prevention of severe manifestations of COVID-19.

The present disclosure is based on the discovery of a new treatment for COVID-19. The inventors of the present disclosure have identified a completely new approach to address the clinical progression of SARS-CoV-2 infections by inhibiting proteases that can lead to inflammatory induced tissue and organ damage during the intermediate and later stages of the disease. A particular advantage of the present therapy is that it should be effective against COVID-19 independent from current or future SARS-CoV-2 variants of concern.

### SUMMARY OF THE INVENTION

The present disclosure provides a new COVID-19 treatment. Thus, in one aspect, the present disclosure provides a polypeptide comprising the sequence of SEQ ID NO:1, or a homologue, fragment or derivative thereof, for use in the treatment of COVID-19. In one embodiment of this aspect, the present disclosure provides a polypeptide comprising the sequence of SEQ ID NO:1, or a homologue, fragment or derivative thereof, for use in the treatment of moderate COVID-19 disease. In one embodiment of this aspect, the present disclosure provides a polypeptide comprising the sequence of SEQ ID NO:1, or a homologue, fragment or derivative thereof, for use in the prevention and/or treatment of severe COVID-19. In one embodiment of this aspect, the present disclosure provides a polypeptide comprising the sequence of SEQ ID NO:1, or a homologue, fragment or derivative thereof, for use in the prevention and/or treatment of disorders or diseases associated with SARS-CoV-2 infection. In one embodiment of this aspect, the present disclosure provides a polypeptide comprising the sequence of SEQ ID NO:1, or a homologue, fragment or derivative thereof, for use in alleviating respiratory distress in a subject infected with the SARS-CoV-2 virus. In one embodiment of this aspect, the present disclosure provides a polypeptide comprising the sequence of SEQ ID NO:1, or a homologue, fragment or derivative thereof, for use in facilitating and accelerating the removal of the need for oxygen supply to a subject infected with the SARS-CoV-2 virus.

In another aspect, the present disclosure provides a method of treating a human subject infected with COVID-19, comprising administering a polypeptide comprising the sequence of SEQ ID NO:1, or a homologue, fragment or derivative thereof. In one embodiment of this aspect, the present disclosure provides a method of treating a human subject with moderate COVID-19 disease, comprising administering a polypeptide comprising the sequence of SEQ ID NO:1, or a homologue, fragment or derivative thereof. In one embodiment of this aspect, the present disclosure provides a method of preventing and/or treating severe COVID-19 in a human subject, comprising administering a polypeptide comprising the sequence of SEQ ID NO:1, or a homologue, fragment or derivative thereof. In one embodiment of this aspect, the present disclosure provides a method of preventing and/or treating disorders or diseases associated with SARS-CoV-2 infection in a human subject, comprising administering a polypeptide comprising the sequence of SEQ ID NO:1, or a homologue, fragment or derivative thereof In one embodiment of this aspect, the present disclosure provides a method of alleviating respiratory distress in a subject infected with the SARS-CoV-2 virus, comprising administering a polypeptide comprising the sequence of SEQ ID NO:1, or a homologue, fragment or derivative thereof In one embodiment of this aspect, the present disclosure provides a method of facilitating and accelerating the removal of the need for oxygen supply to a subject infected with the SARS-CoV-2 virus, comprising administering a polypeptide comprising the sequence of SEQ ID NO:1, or a homologue, fragment or derivative thereof.

In another aspect, the present disclosure provides a pharmaceutical composition comprising a polypeptide comprising the sequence of SEQ ID NO:1, or a homologue, fragment or derivative thereof, for use in the treatment of COVID-19. In one embodiment of this aspect, the present disclosure provides a pharmaceutical composition comprising a polypeptide comprising the sequence of SEQ ID NO:1, or a homologue, fragment or derivative thereof, for use in the treatment of moderate COVID-19 disease. In one embodiment of this aspect, the present disclosure provides a pharmaceutical composition comprising a polypeptide comprising the sequence of SEQ ID NO:1, or a homologue, fragment or derivative thereof, for use in the prevention and/or treatment of severe COVID-19. In one embodiment of this aspect, the present disclosure provides a pharmaceutical composition comprising a polypeptide comprising the sequence of SEQ ID NO:1, or a homologue, fragment or derivative thereof, for use in the prevention and/or treatment of disorders or diseases associated with SARS-CoV-2 infection. In one embodiment of this aspect, the present disclosure provides a pharmaceutical composition comprising a polypeptide comprising the sequence of SEQ ID NO:1, or a homologue, fragment or derivative thereof, for use in alleviating respiratory distress in a subject infected with the SARS-CoV-2 virus. In one embodiment of this aspect, the present disclosure provides a pharmaceutical composition comprising a polypeptide comprising the sequence of SEQ ID NO:1, or a homologue, fragment or derivative thereof, for use in facilitating and accelerating the removal of the need for oxygen supply to a subject infected with the SARS-CoV-2 virus.

### BRIEF DESCRIPTION OF THE SEQUENCE LIST

SEQ ID NO. 1 sets out the primary structure of human elafin.

SEQ ID NO. 2 sets out a preproelafin.

SEQ ID NO. 3 sets out the coding sequence of human elafin.

SEQ ID NO. 4 sets out the repeat contained in the N-terminus of elafin.

SEQ ID NO. 5 sets out the catalytic domain of elafin

### BRIEF DESCRIPTION OF THE FIGURES

FIG. 1: Detailed schematic of the study design.

### LIST OF TABLES

Table 1: Efficacy endpoint(s) of the clinical trial(s).

Table 2: WHO COVID-19 clinical progression scale from the (WHO working group. A minimal common outcome measure set for COVID-19 clinical research. Lancet Infect Dis 2020).

### DETAILED DESCRIPTION OF THE INVENTION

The present disclosure relates to the use of elafin, or a homologue, fragment or derivative thereof, for the treatment of COVID-19. In one embodiment, the present disclosure relates to the use of elafin, or a homologue, fragment or derivative thereof, for the treatment of moderate COVID-19 disease. In one embodiment, the present disclosure relates to the use of elafin, or a homologue, fragment or derivative thereof, for the prevention and/or treatment of severe COVID-19. In one embodiment of this aspect, the present disclosure relates to the use of elafin, or a homologue, fragment or derivative thereof, for the prevention and/or treatment of disorders or diseases associated with SARS-CoV-2 infection. In one embodiment, the present disclosure relates to the use of elafin, or a homologue, fragment or derivative thereof, for alleviating respiratory distress in a subject infected with the SARS-CoV-2 virus. In one embodiment, the present disclosure relates to the use of elafin, or a homologue, fragment or derivative thereof, for facilitating and accelerating the removal of the need for oxygen supply to a subject infected with the SARS-CoV-2 virus.

The following exemplary Embodiments are covered by the present disclosure:
1. A polypeptide comprising the sequence of SEQ ID NO:1 or a homologue, fragment or derivative thereof for use in the treatment of COVID-19 in a human patient.
2. A polypeptide comprising the sequence of SEQ ID NO:1 or a homologue, fragment or derivative thereof for use in the treatment of mild COVID-19 disease in a human patient.
3. A polypeptide comprising the sequence of SEQ ID NO:1 or a homologue, fragment or derivative thereof for use in the treatment of moderate COVID-19 disease in a human patient.
4. A polypeptide comprising the sequence of SEQ ID NO:1 or a homologue, fragment or derivative thereof for use in the prevention of severe COVID-19 disease in a human patient.
5. A polypeptide comprising the sequence of SEQ ID NO:1 or a homologue, fragment or derivative thereof for use in the treatment and/or prevention of disorders or diseases which are associated with SARS-CoV2 infection in a human patient.
6. A polypeptide comprising the sequence of SEQ ID NO:1 or a homologue, fragment or derivative thereof for use in alleviating respiratory distress associated with SARS-CoV2 infection in a human patient.
7. A polypeptide comprising the sequence of SEQ ID NO:1 or a homologue, fragment or derivative thereof for use in facilitating and accelerating the removal of the need for oxygen supply in a human patient infected with the SARS-CoV-2 virus.
8. The polypeptide comprising the sequence of SEQ ID NO:1 or a homologue, fragment or derivative thereof for the use of any one of Embodiments 1 to 7, wherein the patient to be treated had a positive SARS-CoV2-PCR test, is hospitalized, and is Score 4 of the WHO COVID-19 clinical progression scale (no supplemental oxygen provided).
9. The polypeptide comprising the sequence of SEQ ID NO:1 or a homologue, fragment or derivative thereof for the use of any one of Embodiments 1 to 7, wherein the patient to be treated had a positive SARS-CoV2-PCR test, is hospitalized, and is Score 5 of the WHO COVID-19 clinical progression scale (supplemental oxygen provided by mask or nasal prongs).
10. The polypeptide comprising the sequence of SEQ ID NO:1 or a homologue, fragment or derivative thereof for the use of any one of Embodiments 1 to 9, wherein the homologue is defined as having a sequence homology of more than 60%, preferably more than 70%, more than 80%, more than 90%, more than 95%, and even more preferably more than 98%, compared to the polypeptide shown in SEQ ID NO: 1.
11. The polypeptide comprising the sequence of SEQ ID NO:1 or a homologue, fragment or derivative thereof for the use of any one of Embodiments 1 to 9, wherein the derivative differs from the polypeptide shown in SEQ ID NO: 1 or from a homologue and fragment derived therefrom by amino acid modifications, such as glycosylation, PEGylation, biotinylation, cyclization and/or oxidation.
12. The polypeptide comprising the sequence of SEQ ID NO:1 or a homologue, fragment or derivative thereof for the use of any one of Embodiments 1 to 11, wherein the polypeptide comprising the sequence of SEQ ID NO:1 or homologue, fragment or derivative thereof is administered to the patient parenterally (e.g. intravenously, intramuscularly or subcutaneously), by inhalation, orally or intraarterially, preferably intravenously, orally or by inhalation, even more preferably by infusion.

13. The polypeptide comprising the sequence of SEQ ID NO:1 or a homologue, fragment or derivative thereof for the use of any one of Embodiments 1 to 12, wherein the polypeptide comprising the sequence of SEQ ID NO:1 or homologue, fragment or derivative thereof is administered to the patient for a maximum of 10 days, preferably a maximum of 7 days, or until the patient is without oxygen therapy for at least 24 hours (e.g. if earlier than 7 days).
14. The polypeptide comprising the sequence of SEQ ID NO:1 or a homologue, fragment or derivative thereof for the use of any one of Embodiments 1 to 13, wherein the polypeptide comprising the sequence of SEQ ID NO:1 or homologue, fragment or derivative thereof is administered to the patient together with a suitable antiviral or anti-inflammatory therapy, in particular nirmatrelvir plus ritonavir, remdesivir, monoclonal anti-SARS CoV-2 antibodies, preferably casirivimab, imdevimab, sotrovimab, bamlanivimab, etesevimab or tocilizumab; more preferred casirivimab plus imdevimab or sotrovimab; bamlanivimab plus etesevimab; dexamethasone; mechanical ventilation; and/or supplemental oxygen.
15. The polypeptide comprising the sequence of SEQ ID NO:1 or a homologue, fragment or derivative thereof for the use of any one of Embodiments 1 to 14, wherein the polypeptide comprising the sequence of SEQ ID NO:1 or homologue, fragment or derivative thereof is administered to the patient at a dosage of about 10 to about 500 mg/day, preferably about 50 to about 400 mg/day, preferably about 100 to about 300 mg/day, more preferred about 150 to about 250 mg/day, very preferred about 200 mg/day.
16. The polypeptide comprising the sequence of SEQ ID NO:1 or a homologue, fragment or derivative thereof for the use of any one of Embodiments 1 to 15, wherein the polypeptide or homologue, fragment or derivative thereof is a polypeptide having the sequence of SEQ ID NO:1 (human elafin).
17. The polypeptide comprising the sequence of SEQ ID NO:1 or a homologue, fragment or derivative thereof for the use of any one of Embodiments 1 to 16, wherein the patient is at least
18 years of age.
18. The polypeptide comprising the sequence of SEQ ID NO:1 or a homologue, fragment or derivative thereof for the use of any one of Embodiments 1 to 17, wherein the patient is hospitalized for COVID-19 treatment.

19. A method of treating a human patient infected with COVID-19, comprising administering to the patient a polypeptide comprising the sequence of SEQ ID NO:1, or a homologue, fragment or derivative thereof.
20. A method of treating a human patient with mild COVID-19 disease, comprising administering to the patient a polypeptide comprising the sequence of SEQ ID NO:1, or a homologue, fragment or derivative thereof.
21. A method of treating a human patient with moderate COVID-19 disease, comprising administering to the patient a polypeptide comprising the sequence of SEQ ID NO:1, or a homologue, fragment or derivative thereof.
22. A method of preventing severe COVID-19 disease in a human patient, comprising administering to the patient a polypeptide comprising the sequence of SEQ ID NO:1, or a homologue, fragment or derivative thereof.
23. A method of treating and/or preventing disorders or diseases which are associated with SARS-CoV2 infection in a human patient, comprising administering to the patient a polypeptide comprising the sequence of SEQ ID NO:1, or a homologue, fragment or derivative thereof.
24. A method of alleviating respiratory distress associated with SARS-CoV2 infection in a human patient, comprising administering to the patient a polypeptide comprising the sequence of SEQ ID NO:1, or a homologue, fragment or derivative thereof
25. A method of facilitating and accelerating the removal of the need for oxygen supply in a human patient infected with the SARS-CoV-2 virus, comprising administering to the patient a polypeptide comprising the sequence of SEQ ID NO:1, or a homologue, fragment or derivative thereof.
26. The method of any one of Embodiment 19 to 25, wherein the patient to be treated had a positive SARS-CoV2-PCR test, is hospitalized, and is Score 4 of the WHO COVID-19 clinical progression scale (no supplemental oxygen provided).
27. The method of any one of Embodiment 19 to 25, wherein the patient had a positive SARS-CoV2-PCR test, is hospitalized, and is Score 5 of the WHO COVID-19 clinical progression scale (supplemental oxygen provided by mask or nasal prongs).
28. The method of any one of Embodiments 19 to 27, wherein the homologue is defined as having a sequence homology of more than 60%, preferably more than 70%, more than 80%, more than 90%, more than 95%, and even more preferably more than 98%, compared to the polypeptide shown in SEQ ID NO: 1.
29. The method of any one of Embodiments 19 to 27, wherein the derivative differs from the polypeptide shown in SEQ ID NO: 1 or from a homologue or fragment derived therefrom by amino acid modifications, such as glycosylation, PEGylation, biotinylation, cyclization and/or oxidation.
30. The method of any one of Embodiments 19 to 29, wherein the polypeptide comprising the sequence of SEQ ID NO:1 or homologue, fragment or derivative thereof is administered to the patient parenterally (e.g. intravenously, intramuscularly or subcutaneously), by inhalation, orally or intraarterially, preferably intravenously, orally or by inhalation, even more preferably by infusion.
31. The method of any one of Embodiments 19 to 30, wherein the polypeptide comprising the sequence of SEQ ID NO:1 or homologue, fragment or derivative thereof is administered to the patient for a maximum of 10 days, preferably a maximum of 7 days, or until the patient is without oxygen therapy for at least 24 hours (e.g. if earlier than 7 days).
32. The method of any one of Embodiments 19 to 31, wherein the polypeptide comprising the sequence of SEQ ID NO:1 or homologue, fragment or derivative thereof is administered to the patient together with a suitable antiviral or anti-inflammatory therapy, in particular nirmatrelvir plus ritonavir, remdesivir, monoclonal anti-SARS CoV-2 antibodies, preferably casirivimab imdevimab, sotrovimab, bamlanivimab, etesevimab or tocilizumab, more preferred casirivimab plus imdevimab or sotrovimab or bamlanivimab plus etesevimab; dexamethasone; mechanical ventilation; and/or supplemental oxygen.
33. The method of any one of Embodiments 19 to 32, wherein the polypeptide comprising the sequence of SEQ ID NO:1 or homologue, fragment or derivative thereof is administered to the patient at a dosage of about 10 to about 500 mg/day, preferably about 50 to about 400 mg/day, preferably about 100 to about 300 mg/day, more preferred about 150 to about 250 mg/day, very preferred about 200 mg/day.
34. The method of any one of Embodiments 19 to 33, wherein the polypeptide comprising the sequence of SEQ ID NO:1 or a homologue, fragment or derivative thereof is a polypeptide having the sequence of SEQ ID NO:1 (human elafin).
35. The method of any one of Embodiments 19 to 34, wherein the patient is at least 18 years of age.
36. The method of any one of Embodiments 19 to 35, wherein the patient is hospitalized for COVID-19 treatment.

The dosages described in Embodiments 15 and 33 are preferred for intravenous administration. For subcutaneous administration, the dosage would generally be about ⅕^{th} to about 1/10^{th} of the dosages in Embodiments 15 and 33.

According to one aspect of the present disclosure, a method/use of human elafin having the sequence of SEQ ID NO:1 in the treatment of COVID-19 is provided. In a particular aspect, the method comprising administering to a human subject in need thereof a therapeutically effective amount of human elafin (e.g. about 10 to about 500 mg/day, about 50 to about 400 mg/day, about 100 to about 300 mg/day, about 150 to about 250 mg/day, or about 200 mg/day), administered in one or more dosage units (e.g. two 100 mg doses), especially for intravenous administration. For subcutaneous administration, the dosage would generally be about ⅕^{th} to about 1/10^{th} of these quoted dosages. The dosage can also be adapted according to endogenous elafin concentrations in blood or tissue, whereby the attending practitioner is aware of how to determine elafin concentrations in patient samples. As an example, elafin can be measured by ELISA in serum samples. ELISA test kits are described, for example, by Alkemade et al, 1995 (see the Reference list below for details). The elafin level in serum should, for example, be increased by at least 10%, preferably 20%, compared to the pre-existing elafin level.

Elafin, or a homologue, derivative or fragment thereof, may conveniently be administered by infusion to the patient infected with the SARS-CoV-2 virus. Alternatively, elafin, or a homologue, derivative or fragment thereof, may be administered by single or repeated injections or infusions per day to the patient infected with the SARS-CoV-2 virus. Alternatively, elafin, or a homologue, derivative or fragment thereof, may be given as a bolus administration to the patient infected with the SARS-CoV-2 virus. In either case, elafin, or a homologue, derivative or fragment thereof, may be given before or after the patient is diagnosed with COVID-19 if the patient exhibits COVID-19 symptoms.

A “bolus” administration in the present context shall mean an administration, which is carried out only once or twice, preferably once, to achieve the desired effect as described above. In the context of an intravenous bolus, the administration should preferably have a duration of not more than about 60 minutes (infusion), preferably not more than about 30 minutes. However, in an alternative embodiment, the infusion may be continued for up to about 12 hours, or up to about 24 hours.

In the following, any reference to elafin shall automatically also include the herein fragments, homologues and/or derivatives of elafin, unless specifically stated otherwise or the context clearly indicates that elafin having the sequence of SEQ ID NO:1 is intended. The terms “elafin”, “elafin compound” and “the present polypeptide” are used synonymously and are intended to encompass the polypeptide comprising the sequence of SEQ ID NO:1 as well as homologues, derivatives or fragments thereof, unless specifically stated otherwise or the context clearly indicates that elafin having the sequence of SEQ ID NO:1 is intended.

In specific embodiments, it can be necessary to continue the elafin treatment beyond the above indicated time frames. The actual administration time and dosage regime will be determined by the practitioner and will depend on the remaining symptoms.

In one embodiment, the elafin compound is administered intravenously. In another embodiment, the elafin compound is administered orally, or through inhalation. The elafin compound may also be administered subcutaneously.

Suitable dosages of elafin for treating COVID-19 may be determined by a practitioner. A particularly preferred dose in human patients is about 200 mg per day. The administration will be continued in a preferred embodiment for not more than about 10 days, in a more preferred embodiment for not more than about 7 days. Thus, in a typical embodiment, a patient will be treated by intravenous infusion with about 200 mg/day of elafin for not more than about 7 days. In a particular embodiment, elafin is administered by intravenous infusion in two separate applications via an infusion pump, where each application comprising about 100 mg elafin in sodium chloride solution. Preferably, the interval between applications is 12 hours±2 hours.

The treatment with elafin in the present context is unique as it can be started at a relatively earlier phase of the disease, and will last for not more than about 10, or preferably not more than about 7 days. During that time, elafin can reduce the likelihood of progression to more severe disease and limit the number of days of hospitalization.

In a further embodiment, elafin may be given to subjects infected with COVID-19 as a preventive administration, e.g. before diagnosis or shortly after diagnosis of COVID-19, including where the patient exhibits early COVID-19 symptoms but has not been tested for COVID-19. When elafin is given as a preventive administration, it is preferably administered by infusion. Alternatively, administration can be by bolus injection (preferably once or twice, more preferred once) given, for example, intravenously or subcutaneously.

“Before” diagnosis, in accordance with the present disclosure, means an administration which is carried out, for example, within about 10, 9, 8, 7, 6, 5, 4, 3, 2 or 1 hours, preferably 6 hours, more preferably 4 hours, more preferably 2 hours, more preferably within 1 minute to one hour, before the subject is diagnosed by suitable testing as having been infected with COVID.

“Shortly after” diagnosis, in accordance with the present disclosure, means an administration which is carried out, for example, up to several weeks, e.g. up to about 4 weeks, or up to about 2 weeks or of up to about 1 week after diagnosis; preferably within about 7, 6, 5, 4, 3, 2 or 1 days of diagnosis, e.g. within about 3 days, or preferably within about 2 days, or more preferably within about 1 day (e.g. about 6 hours) after diagnosis.

In one embodiment, administration is carried out in patients with a positive SARS-CoV2-PCR test, is hospitalized, and is Score 4 of the WHO COVID-19 clinical progression scale (no supplemental oxygen provided).

In another embodiment, administration is carried out in patients with a positive SARS-CoV2-PCR test, is hospitalized, and is Score 5 of the WHO COVID-19 clinical progression scale (supplemental oxygen provided by oxygen mask or nasal prongs).

“Severe disease” in the context of this disclosure is defined as progression of disease to at least score 6 of the WHO progression scale.

In accordance with the above-described embodiments, the elafin compound can also be used as an advantageous additive for the preparation of medical devices. One example of such a medical device would be a device for mechanical ventilation, for example oxygen masks or nasal prongs or any other inhalation device as known in the art.

SEQ ID NO.: 1 has the following sequence: Ala Gln Glu Pro Val Lys Gly Pro Val Ser Thr Lys Pro Gly Ser Cys Pro Ile Ile Leu Ile Arg Cys Ala Met Leu Asn Pro Pro Asn Arg Cys Leu Lys Asp Thr Asp Cys Pro Gly Ile Lys Lys Cys Cys Glu Gly Ser Cys Gly Met Ala Cys Phe Val Pro Gln.

Elafin was first isolated from the skin of patients with psoriasis, an inflammatory skin disease. It is a soluble protein with 57 amino acids and a molecular weight of about 6 kDa. Cloning of the elafin cDNA revealed that it is synthesized as a 12.3 kDa precursor (117 residues) which is processed intracellularly by cleavage of an N-terminal 22 residue signal sequence to give a 9.9 kDa protein called proelafin (or trappin-2, see below) which is secreted (Molhuizen et al.; Sallenave et al.; Schalkwijk et al.).

Up to now, the analysis of the primary structure of proelafin revealed the presence of two functional domains. The N-terminal domain (residues 1— 60) contains four repeats of the sequence -Gly-Gln-Asp-X-Val-Lys- (SEQ ID NO.: 4) which is characteristic of transglutaminase substrates. The glutamine and lysine residues serve as acyl donors and acceptors, respectively, in the transglutaminase-mediated formation of isopeptide inter-protein cross-links [Molhuizenet al.]. This portion of the molecule is often referred to as the cementoin domain. Tissue transglutaminase is able to cross-link proelafin to a variety of extracellular matrix proteins of the stratum corneum via this domain.

The second domain, consisting of the C-terminal 57 residues, harbours the protease inhibition function of proelafin and is identical to the 6 kDa soluble form of the molecule, i.e. elafin, originally isolated from psoriatic skin. This domain exhibits similarities to members of the whey acidic protein (abbreviated WAP) family in terms of its sequence, protein folding and arrangement of four characteristic disulphide bridges (Tamechika et al.; Tsunemi et al.]. The combination of transglutaminase substrate and WAP domains was subsequently demonstrated in other proteins for which a generic name “the trappin family” was coined. In order to clarify its affiliation with this protein family, the 9.9 kDa proelafin was termed trappin-2. The covalent attachment of proelafin to extracellular matrix proteins has little effect on its ability to inhibit elastase and proteinase-3 (Guyot et al.), suggesting that transglutamination is a means of immobilizing this protease inhibitor in an active form.

The mechanism by which elafin is released from proelafin has not been unequivocally elucidated. Proelafin produced in culture by a type II pneumocyte cell line was processed to elafin only in the presence of serum, indicating that cleavage occurs extracellularly, possibly prior to immobilization by transglutaminase (Sallenave et al.). Consistent with this, tryptase, a mast cell protease was able to selectively release elafin from soluble proelafin (Guyot, Zani, Berger et al.). However, tryptase was inactive with proelafin cross-linked to fibronectin (Guyot et al.). Nevertheless, the fact that acid extracts of psoriatic scales only yield elafin and no cementoin domain, suggests that in vivo elafin is cleaved from proelafin cross-linked to skin matrix proteins. Furthermore, analysis of proelafin breakdown products in the urine revealed only the presence of C-terminal sequences, again pointing to the release of elafin by processing of cross-linked proelafin in vivo (Streit et al.). The enzymes catalyzing elafin detachment from immobilized proelafin still remain to be identified.

The biosynthesis of proelafin is regulated at the transcriptional level and is strongly enhanced in response to the presence of epithelial inflammatory diseases, such as lymphocytic alveolitis and psoriasis. Physical injury, infections, irritation and exposure to ultraviolet radiation also induce elafin expression in the skin. Accordingly, proinflammatory stimuli such as the cytokines IL-1β and TNFα induce the expression of proelafin and elafin in various cultured cells, including respiratory cells and keratinocytes.

The structure of elafin, methods for its preparation, and its use for treating several disorders have also been addressed in the prior art, in particular in European Patent EP 0 402 068, U.S. Pat. Nos. 5,464,822 and 6,245,739 as well as published US Patent Application 2002/0187535. These references also disclose the primary structure of human elafin as depicted in SEQ ID NO.: 1 and its capability to inhibit leukocyte elastase, in particular human leukocyte elastase and porcine pancreatic elastase. Moreover, methods for the preparation of elafin are described in these references. Preparation of derivatives and variants of elafin is also described in EP 0 662 516 and U.S. Pat. No. 5,734,014. All references above are explicitly incorporated herein for reference in their entirety.

Despite the wealth of literature on elafin, and the well-studied effects thereof on elastase, there has, up to now, been no evidence that elafin may be useful in the treatment of COVID-19, in particular any disorder or disease associated with an infection with the SARS-CoV-2 virus.

The invention generally relates to novel uses of polypeptides comprising the sequence depicted in SEQ ID NO: 1, or homologues, derivatives, or fragments of the sequence depicted in SEQ ID NO: 1, for the treatment of medical conditions for which a use of elafin has not yet been contemplated, as defined above and below.

As used herein, the term “homologue” refers to peptides or polypeptides which share a substantial degree of homology on the amino acid level with the sequence of SEQ ID NO: 1 over a certain stretch of its primary structure. In particular, the term “homologue” relates to polypeptides having a sequence (or comprising such sequence) which differs from the sequence depicted in SEQ ID NO: 1 by the substitution (or deletion) of one or more single amino acids. Generally, any amino acid from the sequence depicted in SEQ ID NO: 1 can be deleted or substituted against another amino acid as long as the inhibitory activity on elastase is not lost. Further polypeptides are also included which differ from the sequence of SEQ ID NO: 1 by the insertion of one or more additional amino acids. “One or more” in the above context always refers to 1-50, preferably 1-20, even more preferably 1-10, most preferably 1-5.

Based on the amount of identical amino acids, the sequence homology of a homologue according to the invention is usually more than 60%, preferably more than 70%, more than 80%, more than 90%, more than 95%, and even more preferably more than 98% compared to the polypeptide shown in SEQ ID NO: 1. The degree of amino acid homology may be evaluated by use of suitable computer programs known in the art, such as the GCG program package. A degree of homology, which is used throughout this description interchangeably with “identity”, can be determined also by hybridization techniques, which are well known to a person skilled in the art. The above percentages of identity are thus determined in a preferred embodiment under stringent hybridization conditions. Identity may be measured using sequence analysis software (e.g., ClustalW at PBIL (Pole Bioinformatique Lyonnais) http://npsa-pbil.ibcp.fr).

As is known in the art, a number of different programs can be used to identify whether a nucleic acid or amino acid sequence has identity or similarity to a known sequence. Sequence identity or similarity may be determined using standard techniques known in the art, including, but not limited to, the local sequence identity algorithm of Smith & Waterman, Adv. Appl. Math. 2, 482 (1981), by the sequence identity alignment algorithm of Needleman & Wunsch, J. Mol., Biol. 48,443 (1970), by the search for similarity method of Pearson & Lipman, Proc. Natl. Acad. Sci. USA 85, 2444 (1988), by computerized implementations of these algorithms (GAP, BESTFIT, FASTA, and TFASTA in the Wisconsin Genetics Software Package, Genetics computer Group, 575 Science Drive, Madison, Wis.), the Best Fit sequence program described by Devereux et al., Nucl. Acid Res. 12, 387-395 (1984), preferably using the default settings, or by inspection.

The present invention also provides the inventive polypeptides expressed recombinantly in a suitable host from a corresponding polynucleotide. The present invention particularly provides such polynucleotides, which hybridize under stringent conditions to corresponding polynucleotides. As herein used, the term “stringent conditions” means conditions which permit hybridization between polynucleotides sequences and the polynucleotide sequences of SEQ ID NO: 1 where there is at least about 60%, preferably more than 70%, more than 80%, more than 90%, more than 95%, and even more preferably more than 98% identity.

Suitably stringent conditions can be defined by, e. g., the concentrations of salt or formamide in the prehybridization and hybridization solutions, or by the hybridization temperature, and are well known in the art. In particular, stringency can be increased by reducing the concentration of salt, by increasing the concentration of formamide, and/or by raising the hybridization temperature.

For example, hybridization under high stringency conditions may employ about 50% formamide at about 37° C. to 42° C., whereas hybridization under reduced stringency conditions might employ about 35% to 25% formamide at about 30° C. to 35° C. One particular set of conditions for hybridization under high stringency conditions employs 42° C., 50% formamide, 5x SSPE, 0.3% SDS, and 200 pg/ml sheared and denatured salmon sperm DNA. For hybridization under reduced stringency, similar conditions as described above may be used in 35% formamide at a reduced temperature of 35° C. The temperature range corresponding to a particular level of stringency can be further narrowed by calculating the purine to pyrimidine ratio of the nucleic acid of interest and adjusting the temperature accordingly. Variations on the above ranges and conditions are well known in the art.

Thus, the term homologue also comprises polypeptides which are longer than the sequence of SEQ ID NO: 1 and therefore comprise more amino acids, insofar as a part of their amino acid sequence shares substantial homology with the polypeptide of SEQ ID NO: 1. A polypeptide comprising the sequence depicted in SEQ ID NO: 1 which can be used according to the invention is, for example, the preproelafin shown in SEQ ID NO: 2 (Met Arg Ala Ser Ser Phe Leu Ile Val Val Val Phe Leu Ile Ala Gly Thr Leu Val Leu Glu Ala Ala Val Thr Gly Val Pro Val Lys Gly Gln Asp Thr Val Lys Gly Arg Val Pro Phe Asn Gly Gln Asp Pro Val Lys Gly Gln Val Ser Val Lys Gly Gln Asp Lys Val Lys Ala Gln Glu Pro Val Lys Gly Pro Val Ser Thr Lys Pro Gly Ser Cys Pro Ile Ile Leu Ile Arg Cys Ala Met Leu Asn Pro Pro Asn Arg Cys Leu Lys Asp Thr Asp Cys Pro Gly Ile Lys Lys Cys Cys Glu Gly Ser Cys Gly Met Ala Cys Phe Val Pro Gln (Moihuizen et al.). As can be seen from that sequence, preproelafin comprises an additional N-terminal extension on the N-terminus and is post-translationally cleaved to provide the mature form. Preproelafin (117 amino acids) is first cleaved in proelafin (95 amino acids) and the N-terminal signal peptide (22 amino acids). During the terminal differentiation of keratinocytes (formation of the horny layer), proelafin becomes crosslinked to cornified envelope proteins by epidermal transglutaminase. The mature elafin is apparently released from these cornified envelope proteins by a yet unknown mechanism and can be extracted from horny layers of human skin, particularly from scales of patients suffering from psoriasis.

According to the present invention, the term “fragment” refers to any biologically active portion of the polypeptide in SEQ ID NO: 1 having the desired enzymatic activity on the inhibition of elastase. A fragment of elafin can consist of an amino acid sequence differing from the amino acid sequence in SEQ ID NO.: 1 by the deletion of one or more amino acids at the N-terminus and/or C-terminus. For example, a fragment according to the invention may lack amino acid residue (s) 1, 1-2, 1-3, 1-4, 1-5, 1-6, 1-10 or 1-20 at the N-terminus of the polypeptide. Similarly, it may lack the corresponding residues at the C-terminus. Also, the elafin fragment can consist of the N-terminus of elafin. In a particular embodiment, the fragment retains the amino acid sequence of SEQ ID NO.: 1 between (and including) Cys 16 and Cys 53 of SEQ ID NO.: 1 (i.e. Cys 76 to Cys 113 of SEQ ID NO.: 2). This fragment is reflected in SEQ ID NO.: 5. This part of the depicted sequences contains the elastase inhibitory domain and thus the key part responsible for the biological activity of elafin. A high number of variations in both the N-terminal as well as the C-terminal part can be carried out, or these parts can even be deleted completely, without any appreciable decrease in the biological activity of elafin. However, even the inhibitory domain can potentially comprise certain variations or deletions as long as the biological activity of elafin is retained, as explained in more detail below.

Moreover, a fragment may differ from the amino acid sequence in SEQ ID NO: 1 by lacking amino acid residues at both the N-terminus and C-terminus. For example, a fragment may consist of amino acids 6-30 of the polypeptide shown in SEQ ID NO: 1. Further comprised by the invention is the use of homologues of the fragments of the polypeptide shown in SEQ ID NO: 1.

The term “derivatives” refers to peptides or polypeptides which differ from the polypeptide shown in SEQ ID NO: 1 or from the homologs and fragments derived therefrom by well-known amino acid modifications, such as glycosylation, PEGylation, biotinylation, cyclization and/or oxidation. PEGylation is particularly preferred. Methods to provide such derivatives are very well known to the person skilled in the art. One exemplary method for PEGylation is described below.

It is preferred that all above described fragments, homologues and derivatives of the invention as compared to the polypeptide of SEQ ID NO:1 have the function of being useful for the treatment of COVID-19, in particular any disorder or disease associated with an infection with the SARS-CoV-2 virus, a respective method of treatment for COVID-19 and composition comprising elafin, for use in said treatment, and additionally or concomitantly for the use in the treatment and/or prevention of complications associated with COVID-19 and/or progression to severe disease upon infection with SARS-CoV-2 virus.

All fragments, homologues and/or derivatives of the present disclosure should retain the biological activity of elafin. In the present context, “biological activity of elafin” is defined as being able to inhibit human leukocyte elastase. Human leukocyte elastase inhibition activity should be maintained with at least 50%, preferably at least 60%, 70%, 80%, 90%, 95%, 98%, 99% or 100%. The human leukocyte elastase inhibition can be assayed by a well-known inhibition assay. One human leukocyte elastase inhibition assay is described in the following:

Inhibition of human leukocyte elastase can be determined using the synthetic peptide methoxy-succinyl-alanyl-alanyl-prolyl-valyl-p-nitroanilide (SEQ ID NO.:6) (AAPVpNA, Sigma): 100 μL sample and 100 μL human leukocyte elastase (Elastin Products Corporation, Pacific, Mo., USA) (200 ng/mL) in assay buffer (0.1 M, HEPES, 0.5 M NaCl, 10% Me₂SO, pM 7.5) are incubated for 30 min. at room temperature before adding 800 μL 0.5 mM substrate in assay buffer. Changes in absorption at 405 nm can be followed up to 1 h and %-inhibition can be calculated from remaining activity and controls. Kᵢ can be determined in the same system, using 2 mM substrate and 1 μg/ml human leukocyte elastase.

In an even more preferred embodiment, the fragments, homologues and/or derivatives of the present disclosure additionally retain further biological activities of elafin, namely inhibition of serine proteinase 3, inhibition of formation of neutrophil exosomes, inhibition of neutrophil extracellular traps, inhibition of transcription factors AP-1 and NF-κB, and/or inhibition of troponin I and/or T. Assays to measure any of these activities are part of the general knowledge of the person skilled in the art. The respective inhibitory activity should be maintained with at least 50%, preferably at least 60%, 70%, 80%, 90%, 95%, 98%, 99% or 100%.

The polypeptides of the present disclosure can be administered together with other agents for the treatment of COVID-19, e.g. a suitable antiviral or anti-inflammatory therapy, in particular nirmatrelvir plus ritonavir, remdesivir, monoclonal anti-SARS CoV-2 antibodies, preferably casirivimab, imdevimab, sotrovimab, bamlanivimab, etesevimab or tocilizumab; more preferred casirivimab plus imdevimab or sotrovimab; bamlanivimab plus etesevimab; dexamethasone, mechanical ventilation and/or supplemental oxygen. Further optional co-treatments are anakinra, siltuximab and sarilumab. “Together with” in this context means that the polypeptide of the disclosure is administered simultaneously with the other agent(s), or after the other agent(s) has been administered, or before the other agent(s) is administered. An administration before or after the administration of the other agent can be an administration any suitable time up to about 1 week before or after the administration of the other agent, for example within about 1 minute to about 1 hour (e.g. within about 1 minute, about 5 minutes, about 10 minutes, about 30 minutes, or about 1 hour), or within about 2 hours, within about 5 hours, within about 12 hours, within about 24 hours, within about 2 days, within about 3 days or within about a week before or after the administration of the other agent.

The polypeptides of the invention can be obtained as described in the prior art (see, for example, EP 0 402 068) or can be prepared by recombinant expression of the coding sequence as described by Sallenave et al.). The coding sequence is also provided in SEQ ID NO: 3 (aggccaagct ggactgcata aagattggta tggccttagc tcttagccaa acaccttcct gacaccatga gggccagcag cttcttgatc gtggtggtgt tcctcatcgc tgggacgctg gttctagagg cagctgtcac gggagttcct gttaaaggtc aagacactgt caaaggccgt gttccattca atggacaaga tcccgttaaa ggacaagttt cagttaaagg tcaagataaa gtcaaagcgc aagagccagt caaaggtcca gtctccacta agcctggctc ctgccccatt atcttgatcc ggtgcgccat gttgaatccc cctaaccgct gcttgaaaga tactgactgc ccaggaatca agaagtgctg tgaaggctct tgcgggatgg cctgtttcgt tccccagtga gagggagccg gtccttgctg cacctgtgcc gtccccagag ctacaggccc catctggtcc taagtccctg ctgcccttcc ccttcccaca ctgtccattc ttcctcccat tcaggatgcc cacggctgga gctgcctctc tcatccactt tccaataaag agttccttct gctccaaaaa aaaaaaaaaa aaaaaaaaaa aaa).

The polypeptides, homologues, derivatives and fragments as defined herein may be used as obtained or purified in a known and appropriate manner and formulated into pharmaceutical compositions.

In the present disclosure, pharmaceutical compositions ordinarily refer to agents for treating or preventing, or testing and diagnosing, diseases. For example, formulation into pharmaceutical compositions may be achieved by admixture with a pharmaceutically acceptable diluent or carrier. Administration may be by way of various routes known in the art and defined herein in more detail.

The pharmaceutical compositions disclosed herein can be formulated by methods known to those skilled in the art. For example, they can be used parenterally, in the form of injections of sterile solutions or suspensions including water or other pharmaceutically acceptable liquid. For example, such compositions may be formulated by mixing in the form of unit dose required in the generally approved medicine manufacturing practice by appropriately combining with pharmaceutically acceptable carriers or media, specifically with sterile water, physiological saline, vegetable oil, emulsifier, suspension, surfactant, stabilizer, flavoring agent, excipient, vehicle, preservative, binder, or such. In such formulations, the amount of active ingredient is adjusted to obtain an appropriate amount in a pre-determined range.

Sterile compositions for injection can be formulated using vehicles such as physiological saline, e.g. 0.9% aqueous NaCl solution in distilled water for injection, according to standard formulation practice.

Aqueous solutions for injection include, for example, physiological saline and isotonic solutions containing dextrose or other adjuvants (for example, D-sorbitol, D-mannose, D-mannitol, and sodium chloride). It is also possible to use in combination appropriate solubilizers, for example, alcohols (ethanol and such), polyalcohols (propylene glycol, polyethylene glycol, and such), non-ionic surfactants (polysorbate 80(TM), HCO-50, and such).

Oils include sesame oil and soybean oils. Benzyl benzoate and/or benzyl alcohol can be used in combination as solubilizers. It is also possible to combine buffers (for example, phosphate buffer and sodium acetate buffer), soothing agents (for example, procaine hydrochloride), stabilizers (for example, benzyl alcohol and phenol), and/or antioxidants. Appropriate ampules are filled with the prepared injections.

The pharmaceutical compositions disclosed herein are preferably administered parenterally. For example, the compositions may be in the dosage form for injections, transnasal administration, transpulmonary administration, or transdermal administration. For example, they can be administered systemically or locally by intravenous injection, intramuscular injection, intraperitoneal injection, subcutaneous injection, or such. As described above, preferred administration routes are infusion, oral administration or administration by inhalation.

For intravenous injection and infusion, a suitable well known sterile carrier material can be used. One typical example would be a saline solution. Particularly preferred is about a 0.9% NaCl solution. Even more preferred is about 5 to about 15 mg/ml of elafin in the NaCl solution, preferably in about 0.9% NaCl solution. Most preferred is about 10 mg/ml elafin in about 0.9% NaCl solution. All of the above formulations may be conveniently stored prior to use. Administration may then conveniently take place following dilution to about 250 ml with saline solution.

For oral administration of elafin, a preferred formulation is elafin, preferably PEGylated elafin, which is coated with a polymer which is pH responsive, i.e. is insoluble in acid but dissolves in a mildly alkaline environment, i.e. pH 7 or above, which makes it optimal for colonic delivery. Eudragit FS30D polymer is one example of such a polymer, but the person skilled in the art is well aware of further possibilities. The coated elafin can optionally be packaged into microparticles which can prevent leakage of elafin in acidic, aqueous solution. PEGylation can be achieved by well-known methods; one example is conjugation with methoxyl-PEG12 (New England Peptide Company).

For inhalation, typical carrier materials can be used which are well known to the person skilled in the art. For example, lactose or spray granulated mannitol are suitable carrier materials for use in dry powder inhalation. Alternatively, a mechanically produced aerosol without carriers can be used. Intratracheal instillations of elafin in saline (e.g. 0.9% aqueous NaCl solution) are another option.

Administration methods can alternatively be appropriately selected in consideration of the patient's age and symptoms. The dose may be, for example, from 0.0001 to 1000 mg/kg for each administration. Alternatively, the dose may be, for example, from 0.001 to 100,000 mg per patient. In one embodiment, the dose is about 0.1 to about 10 mg/kg. In one embodiment, the dose is about 10 to about 500 mg per day, e.g. about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 or 500 mg per day. In a particular embodiment, the dose is about 200 mg per day, preferably given as an intravenous dose. For subcutaneous administration, the dose is about ⅕^{th} to about 1/10^{th} lower than indicated above. However, the present disclosure is not limited by the numeric values described above. The doses and administration methods vary depending on the patient's weight, age, symptoms, and such. Those skilled in the art can set appropriate doses and administration methods in consideration of the factors described above.

Amino acids contained in the amino acid sequences disclosed herein may be post-translationally modified. For example, the modification of an N-terminal glutamine (Gln) residue into a pyroglutamic acid (pGlu) residue by pyroglutamylation is well-known to those skilled in the art. Naturally, such post-translationally modified amino acids are included in the amino acid sequences disclosed herein.

The pharmaceutical compositions will be formulated according to the mode of administration to be employed. For example, when the composition is to be administered by inhalation via the oral route or intra-nasally, the composition may be formulated as a powdered aerosol, and when the composition is to be administered by way of injection it may be formulated as a sterile solution or suspension.

Suitable diluents including aqueous solutions and additives, such as buffers and surfactants may be added. Pharmaceutical compositions of the present invention also include controlled release formulations. For example, the polypeptides of the present invention may be encapsulated in a biodegradable polymer or may be dispersed in a matrix of such a polymer, so that the polypeptide is released as the degradation of the polymer matrix proceeds. Suitable biodegradable polymers for use in sustained release formulations include polyesters which gradually become degraded by hydrolysis when placed in an aqueous, physiological environment. A particular pharmaceutical composition which provides for extended release of a polypeptide is described in European Patent No. 0058481. In this composition a polylactide is employed, and when placed in an aqueous physiological-type environment, the polypeptide is released from the composition in a continuous manner until essentially all of the polypeptide has been released. Such polymers therefore offer the advantage of a highly localised target area, thus minimizing dosage and any potential side effects.

Elafin may be administered systemically or by use of microspheres incorporated, for example, into a medical device, e.g. into an oxygen mask or onto a nasal prong, which release the elafin in a controlled manner. The elafin can also be used in the form of elafin-containing polymers that release elafin in a controlled manner. The high stability of elafin to ethylene oxide sterilization is a further important aspect for its therapeutic application in this context, especially if is to remain active as a slow-release drug or as a component in a medical device coating. The biocompatibility of elafin has been proven.

The examples described below refer to the compound Tiprelestat. This is the INN name of elafin.

The present disclosure can provide for a significant and positive impact on various levels. These can be divided into the following categories:

### Societal

Making Tiprelestat available for the treatment of patients suffering from COVID-19 to mitigate disease complications, such as inflammatory complications, is of significant public interest. Inflammatory complications are a major cause of debilitating illness and pain in patients and are potentially life-threatening, imposing stress on health infrastructure and intensive care units. Hence, inflammatory-induced morbidity has a strong negative impact on the quality of life of patients which affects their participation in the society and their productivity. The treatment and care of patients with COVID-19-induced complications and potential hospital re-admissions represents a strong burden with regard to the workload of hospital personnel, especially the intensive care physicians and nurses and the hospital budget. Family and relatives are also strongly involved in the care of patients suffering from these post-inflammatory COVID-19 induced complications.

### Economic

Severe and critical COVID-19 patients generate average complication costs of EUR 38,500 each. However, deviations from this average can be significant with 10% of all patients requiring ventilation causing costs of more than EUR 85,000 each (Arztezeitung 2020). In this calculation, the costs for society (lockdown costs, outpatient care, productivity etc.) as well as for hospital re-admission are not included.

### Sustainability

Treatment according to the present disclosure can serve to drastically lower the global burden of communicable diseases, which constitutes one of the major challenges for sustainable development in the twenty-first century according to the WHO Goals for Sustainable Development (WHO Resolution General Assembly 66/288 Health and population 141). It further aims to reduce high unsustainable and avoidable burdens on healthcare systems by lowering the resources required for intensive care and thereby freeing up available capacities in times of crisis.

### EXAMPLES

### Example 1

### Background

The objective of the clinical program (“the Project”) is the clinical development of a new therapeutic approach in the treatment of COVID-19, particularly the treatment of moderate disease, especially, the treatment of hospitalized subjects with a score of 5 according to the WHO COVID-19 clinical progression scale, and the prevention of progression to severe COVID-19 disease.

This objective necessitates the implementation of a two-pronged approach covering the conduct of a phase Ib/II multi-centric, randomized, placebo-controlled, double-blinded clinical trial as well as the scale-up of production processes and manufacture of cGMP study material.

In the context of the clinical phase Ib/II trial, the efficacy and safety/tolerability of Tiprelestat is assessed.

This trial is carried out in approximately 10 to 15 centers in Germany with a total patient population of 296 (148 active drug/148 placebo).

Hospitalized SARS-CoV-2-PCR positive patients corresponding to Score 5 according to the WHO COVID-19 progression scale are treated with an infusion of 200 mg Tiprelestat per day. Thus, 100 mg Tiprelestat is administered twice per day by infusion. Each infusion administers 100 mg Tiprelestat over about 30 minutes. The second infusion is given 12 hours±2 hours after the first infusion. The treatment period is 7 days or until the patient has been without oxygen therapy for at least 24 hours, if earlier.

Key endpoints of this clinical trial are as follows. The primary efficacy endpoint relates to the number of days the patient receives any oxygen support. Secondary efficacy endpoints encompass the proportion of patients who progress to severe disease (Score 6 or greater according to the WHO COVID-19 progression scale), time to first occurrence of severe disease, duration of severe disease, duration of ICU stay, duration of dyspnea, duration of fatigue, 28-day mortality and 90-day mortality. Example 6 hereinafter provides further information regarding clinical endpoints.

### Example 2

### Study Medication—Preparation of Tiprelestat

The drug substance Tiprelestat is manufactured in accordance with cGMP (current Good Manufacturing Practice). Briefly, Hansenula polymorpha cells containing the expression vector coding for Tiprelestat are grown in a 3,000 L, concentrated and washed by microfiltration. The permeate containing Tiprelestat is purified from the permeate by a two-step chromatography. Tiprelestat is concentrated and formulated to give a solution of 10 mg/mL in 0.9% NaCl by ultrafiltration and diafiltration before sterile filtration. The whole batch is filled into in 5 mL aliquots in glass vials, labeled and frozen as drug product to -20° C. The vials are delivered frozen to the study sites.

The Tiprelestat concentrate for infusion (50 mg Tiprelestat in 5mL 0.9% saline) is packaged in 10 mL injection vials with vial injection stoppers and flip off seals. Vials are packed in labelled boxes. Injection vials and boxes are labelled according to European and local national requirements. The pharmacy delivers the study medication to the investigator as infusion bags containing 100 mg Tiprelestat in 100 mL 0.9% sodium chloride solution or placebo (100 mL 0.9% sodium chloride solution). For the labelling of the study medication infusion bags, the pharmacies receive written instructions, and an adequate training is performed before study start at the respective site (i.e. study initiation visit).

### Example 3

### Scientific and/or Technical Objectives of the Project

Extensive preclinical and clinical data for the use of Tiprelestat in several indications has been obtained (please also refer to Example 6), and its use in the context of treating SARS-CoV-2 infection (specifically moderate disease), including reducing the percentage of hospitalized patients progressing from moderate to severe disease, is demonstrated in the Project. The degree of severity of the SARS-CoV-2 infection is as defined by the Robert-Koch-Institute (RKI) (RKI: Hinweise zu Erkennung, Diagnostik and Therapie von Patienten mit COVID-19, 16.07.2021) and measured in line with the COVID-19 WHO ordinal scale (WHO working group. A minimal common outcome measure set for COVID-19 clinical research. Lancet Infect Dis 2020).

### Example 4

### Development Stage of the Project

### Key Considerations

As outlined, the clinical course of COVID-19 is highly variable, ranging from asymptomatic to multi-organ failure and death. The current efforts (excluding vaccinations) to prevent and treat COVID-19 are so far limited to symptomatic treatments, all of which are insufficient to substantially reduce the rate of progression to severe disease and lethality.

COVID-19 is a generalized disease affecting almost all organs. The first pathologic investigations showed the presence of a generalized involvement of the vasculature in form of an endotheliitis with SARS-CoV-2 detection in the vascular endothelium (Varga et al.). This is followed by a proliferation of vascular endothelial and smooth muscle cell proliferation (Suzuki et al.) leading to low vascular perfusion and occlusions (Suzuki et al.). This also seems to explain why COVID-19 has a high rate of involvement of different organs, of which the involvement of lung, heart, kidneys, and liver are dominant (Zhou et al.). The severity of these organ involvements, in particular the development of ARDS, acute kidney injury and cardiac complications, are associated with a high risk of death.

### Safety of Tiprelestat

Please refer to Example 6.

### Pharmacokinetics

In rats, the pharmacokinetics of 99mTc-labeled elafin (99mTc-Elafin) was instigated in blood and individual organs after bolus intravenous injection using the single photon emission tomography (SPECT). The terminal elimination half-life (t1/2 (3) of 99TC-elafin in blood was 200 minutes. 99mTc-elafin predominantly accumulated in the kidney reaching a maximum of 8.5%±0.1% of the injected dose per gram (ID/g) at 5 min post injection (p.i.) and decreased only slowly during 24 h. In contrast, the initially high radioactivity recorded in the other organs rapidly decreased parallel to the radioactivity detected in blood. The blood kinetics fits to a two compartment kinetics model. The radioactivity in the dissected kidney was 4.98±1.24%ID/g 24 h p.i, while in other organs, including the brain, no accumulation of 99mTc-elafin was detected. At this time point 30% of the detected radioactivity in the kidney was identified to be not metabolized 99mTc-Elafin (Kaschwich et al.).

### Dose Range for Clinical Use

In a Phase I clinical study single intravenous doses of 20 to 400 mg Tiprelestat and in two clinical Phase II studies intravenous doses of 200 mg Tiprelestat were well tolerated.

In the present study, a daily infusion dosing of 200 mg Tiprelestat (two 100 mg infusions per day) up to 7 days is administered. Safety of the extension of the dosing interval will be evaluated in the initial Phase lb part of the study (Please also refer to Example 6).

### Example 5

### Characterization of Elafin

### Stage of Development

One phase I and two phase II randomized double-blind placebo-controlled clinical trials in a surgery program demonstrated that intravenous application of Tiprelestat is well tolerated and established the proof of concept of Tiprelestat for the preventive use in surgery.

No major safety concerns were observed and Tiprelestat's organ protective effect was confirmed on the basis of a clinically relevant endpoint, the length of ICU stay after esophageal cancer surgery, associated with a trend towards lower kidney, liver and heart organ damages. The clinical trials in the surgery development program established an intravenous single dosing scheme with perioperative administration via infusion.

### Pre-Clinical Investigations

Tiprelestat has been subjected to numerous preclinical investigations in animals in a variety of disease models. Tiprelestat suppresses reperfusion injury occurring after heart transplantation (Cowan et al.), myocardial infarction (Tiefenbacher et al. and Ohta et al.), and skeletal muscle ischemia (Crinnion et al.). Tiprelestat is able to reduce ventilator induce injury (Hilgendorff et al. 2011, Han et al., Alcazar et al.) . Furthermore, transgenic animals expressing human elafin in the cardiovascular system became much more resistant to viral myocarditis (Zaidi et al. 1999), ventilator injury (Hilgendorff et al. 2012), vascular injury (Zaidi et al. 2000), angioplasty (Barolet et al.), vein graft degeneration (O'Blenes et al.) as well as pulmonary arterial hypertension induced by hypoxia (Zaidi et al. 2002).

### In Vivo Data in Disease Models

In vivo animal models of SARS-CoV-2 infection and transmission as well as the characterization of the immunological response are established. This is in contrast to animal models of COVID-19 to depict the severe clinical course in humans on the basis of an experimental SARS-CoV-2 infection (Rosa et al.). In addition, animal models of experimental SARS-CoV-2 infection cannot adequately depict the factors that are decisive for severe disease progression in humans, such as age and comorbidities.

In vivo data on the effectiveness of elafin in preventing severe or fatal courses of COVID-19 is collected through the effect of elafin on the organ manifestations that are considered relevant to the severity of the COVID-19, which include at least:
development of pulmonary-arterial hypertension with secondary heart failure;
development of acute respiratory failure (ARDS);
development of myocardial damage, especially myocarditis and heart attack;
ventilation damage caused by prolonged mechanical ventilation and
the development of cytokine release syndrome, in which elevated blood levels of IL-6, IL-10 and TNF-α are associated with severe disease.

Data demonstrating the effect of Tiprelestat (human recombinant elafin) in various in vivo models are described in:

Zaidi at al. 2002; Nickel et al. 2015; Wang et al. 2017; Alam et al.; Zaidi et al. 1999; Ohta et al.; Tiefenbacher et al.; Shavadia et al.; Han et al.; Hilgendorff et al. 2011); and Hilgendorff et al. 2012;

In addition, it has been observed that mortality is more than twice as high in acute lung failure (ARDS) in patients with below-average elafin blood levels - the most important lethal COVID-19 manifestation.

### Example 6

### Clinical Trial

### Study Title

COVID-19: Efficacy and safety of Tiprelestat for the treatment of COVID-19 Short title: COMCOVID-Study

### Study Design and Endpoints

### Study Design

The objective of this study is the assessment of efficacy and safety of Tiprelestat in treating SARS-CoV-2 infection (specifically moderate disease), including reducing the percentage of hospitalized patients progressing from moderate to severe disease, and treatment of organ complications in SARS-CoV-2 infected hospitalized patients with COVID-19 prior to the need of high flow oxygen or artificial ventilation.

The study is designed as a national, randomized, placebo-controlled, double-blinded, multicenter parallel-group Phase Ib/II clinical study. 296 patients are randomized to a treatment group or placebo group from approximately 10 to 15 German study centers. Patients eligible for participation in this clinical study are those who are hospitalized for COVID-19 treatment following a positive SARS-CoV2-PCR test, and are receiving oxygen therapy by mask or nasal prongs (Score 5 according to the WHO COVID-19 progression scale). Tiprelestat or placebo is administered by infusion for a maximum of 7 days, or until the patient is without oxygen therapy for at least 24 hours, if earlier. However, the first patient (the “sentinel patient”) will be treated open label with Tiprelestat 200 mg daily for at least 4 treatment days. The sentinel patient [see also Example 6 (a) below] receives Tiprelestat open-label to verify safety and demonstrate an initial indication of efficacy. There is no restriction on concomitant treatments.

The study is designed as a placebo-controlled Phase Ib/II study, where the first 16 patients in both treatment groups, together with the sentinel patient, are regarded as a Phase Ib group for obtaining safety and Tiprelestat kinetic data. Allocation to the study groups (Tiprelestat versus Placebo) is performed by the use of a randomization list. Thereby equal distribution of both groups within the first 32 patients (Phase Ib part of the study) is ensured. Randomization is carried out after the pre-study examination. The subjects will be assigned to the random numbers in ascending order.

The allocation to the treatment group is determined by the randomization list.

The sentinel patient and the subsequent pilot set of further 12 patients are observed five times daily, with an interval of 4 to 7 hours, for their vital signs and SpO2 level. The “PK-set of patients”, including the sentinel and the pilot set of patients, will undergo more frequent daily assessments of safety laboratory tests than the subsequent “regular” patients of this study. In subsequent “regular” patients, assessment and documentation of SpO2% will be done twice daily from Day 2 to Day 29 or until the patient has been without oxygen therapy for at least 24 hours, if earlier. Assessment and documentation of SpO2% is performed in the morning and in the afternoon/evening.

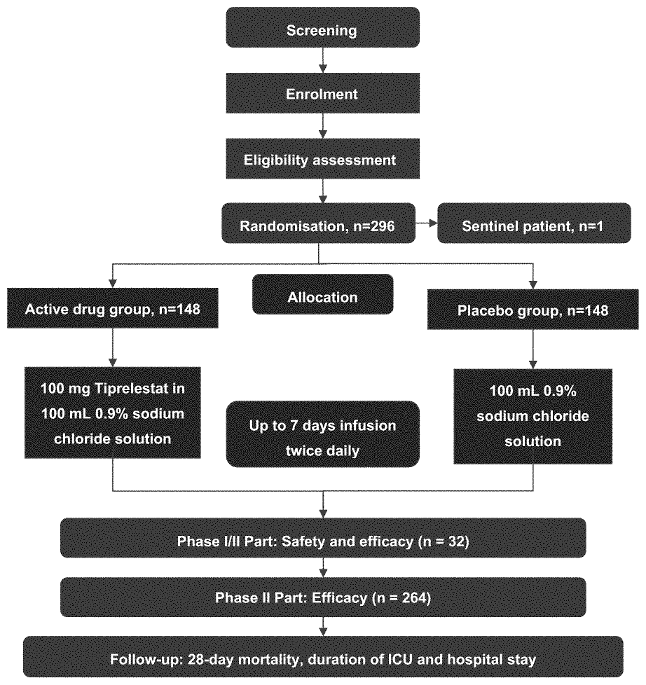

A detailed schematic of the study design can be found in FIG. 1.

### Primary and Secondary Endpoints:

TABLE 1
Efficacy endpoints of the clinical trial

| Efficacy endpoint | Description |
|---|---|
| Primary efficacy | Number of days* with any oxygen support |
| endpoint | (≥Score 5 according to the WHO |
| | COVID-19 progression scale) |
| Secondary | Proportion of patients [n/N] with |
| efficacy | progression to severe disease according |
| endpoints | to the WHO COVID-19 clinical progression |
| | scale (score ≥ 6)* |
| | Time to first occurrence of severe disease |
| | (score ≥ 6) according to the WHO COVID-19 |
| | clinical progression scale* |
| | Number of days of severe disease (score ≥ 6) |
| | according to the WHO COVID-19 clinical |
| | progression scale* |
| | Number of days in ICU* |
| | Number of days with dyspnea* |
| | Number of days with fatigue* |
| | 28-day mortality [n/N] |
| | 90-day mortality [n/N] |
| Further | Proportion [n/N] and number of days |
| efficacy | with assisted mechanical |
| endpoints | ventilation or ECMO* |
| | Proportion [n/N] and number of days with |
| | acute kidney injury (AKI), requirement of |
| | hemofiltration or dialysis techniques or |
| | active diuresis (i.e. intravenous diuresis)* |
| | Proportion [n/N] and number of days with |
| | catecholamine administration* |
| | Proportion [n/N] and number of days with |
| | new onset myocardial arrhythmia* |
| | Proportion [n/N] and number of days with |
| | any other deterioration symptoms and |
| | complications of COVID-19* |
| Safety | Proportion [n/N] of adverse events (AEs) |
| | and adverse drug reactions (ARs) |
| | Safety laboratory data |

*Time frame: Day 1 after randomization to Day 29 or Day 1 after randomization to Day of Discharge, if earlier

TABLE 2
WHO COVID-19 clinical progression scale (WHO working
group. A minimal common outcome measure set for COVID-19
clinical research. Lancet Infect Dis 2020)

| Patient State | Descriptor | Score |
|---|---|---|
| Uninfected | Uninfected; no viral RNA detected | 0 |
| Ambulatory - | Asymptomatic; viral RNA detected | 1 |
| mild | Symptomatic; independent | 2 |
| disease | Symptomatic; assistance needed | 3 |
| Hospitalized - | No oxygen therapy (if hospitalized | 4 |
| moderate | for isolation only, record status | |
| disease | as for ambulatory patient) | |
| | Oxygen by mask or nasal prongs | 5 |
| Hospitalized - | Oxygen by non-invasive ventilation | 6 |
| severe | or high flow | |
| Disease | Intubation and mechanical ventilation, | 7 |
| | pO2/FiO2 ≥ 150 or SpO2/FiO2 ≥ 200 | |
| | Mechanical ventilation pO2/FIO2 < 150 | 8 |
| | (SpO2/FiO2 < 200) or vasopressors | |
| | Mechanical ventilation pO2/FiO2 <150 | 9 |
| | and vasopressors, dialysis, or ECMO | |
| Dead | Death | 10 |

In the Phase Ib part of the study, in 33 patients (including the sentinel patient) blood samples are analyzed in a centralized laboratory for: hemoglobin, hematocrit, red blood cell count (RBC), white blood cell count (WBC), white differential count, platelet count, mean corpuscular hemoglobin (MCH), mean corpuscular hemoglobin concentration (MCHC), mean corpuscular volume (MCV), normalized prothrombin ratio (INR) and activated partial thromboplastin time (APTT), alkaline phosphatase (AP), aspartate aminotransferase (AST), alanine aminotransferase (ALT), gamma-glutamyl transpeptidase (GGT), lactate dehydrogenase (LDH), amylase, creatine phosphokinase (CK), glucose, creatinine, uric acid, urea, total bilirubin, total protein, sodium, potassium, calcium, chloride. Furthermore, elafin serum concentrations are determined at day one and at the end of Tiprelestat administration.

### Subjects/Population(s)

### Inclusion Criteria:

1. written informed consent;
2. positive SARS-CoV2-PCR test (not older than ten days) confirmed by a laboratory;
3. hospitalized for COVID-19 treatment;
4. Score 5 on the WHO COVID-19 clinical progression scale
5. male or female, 18 years of age or older.

### Exclusion Criteria:

1. lifetime expectancy of 2 days or less as judged by the investigator;
2. malignant disease requiring chemotherapy, radiation therapy and/or immune therapy at the time of enrolment;
3. patient requires dialysis;
4. concomitant respiratory disease requiring supplemental oxygen therapy, apart from COVID-19;
5. pregnant or breastfeeding females (all female subjects deemed of childbearing potential by the investigator must have negative pregnancy test at screening);
6. current or previous participation within the past 30 days in another interventional clinical trial with an investigational medicinal product;
7 known to be or suspected of being unable to comply with the clinical trial protocol;
8. legal incapacity and/or other circumstances rendering the patient unable to understand the nature, scope and possible impact of the clinical trial;
9. patient in custody by judicial or official order; evidence of an uncooperative attitude;
10. patient who is a member of staff of the trial center, staff of the sponsor or CRO, the investigator or a close relative of the investigator

### Conduct

### Description and Assignment of Intervention

### Interventions

The patients receive either 100 mg Tiprelestat in 100 mL 0.9% sodium chloride solution twice per day as infusions over up to 7 days or 100 mL 0.9% sodium chloride solution alone twice per day as infusions over up to 7 days after admission to the hospital.

### Allocation

Allocation to the treatment groups is performed by randomization at hospital admission.

### Blinding

The study is performed in a double-blind fashion. The investigator and the study staff (except the responsible pharmacist and his/her designee), the subjects, the monitors and the sponsor remain blinded to the treatment allocation until closure of the clinical database. Randomization is performed by the CRO. The randomization code is kept strictly confidential. It is accessible only to authorized persons who are not involved in the conduct and analysis of the study, until the time of unblinding. Since Tiprelestat is colorless and odorless, it is not possible to distinguish between verum and placebo infusions during the conduct of the study.

### Unblinding

Unblinding is performed by the clinical trial statistician after all clinical data have been entered into a curated database which will then be locked.

### Adverse Event Reporting

### Classification of Medically Abnormal Findings

In a first assessment step, the investigator must assess each untoward medical occurrence in a case-to-case decision with respect to relationship to the natural course of a SARS-CoV-2 infection.

All SARS-CoV-2 infections related untoward medical occurrences are documented as complications of SARS-CoV-2 in study specific evaluation forms but will not be documented as AE. All other untoward medical occurrences are documented as adverse events (AEs).

### Assessment and Analysis of AEs

The Investigator reviews the patient's records (including e.g. laboratory and diagnostic reports) to identify the occurrence of AEs until Day 29 or until Day of Discharge, if earlier. Additional information may be obtained from hospital staff and verbal questioning of the patient if the patient is able to communicate.

AEs are recorded in study specific evaluation forms which have to be transcribed into the eCRF using a recognized medical term or diagnosis that accurately reflects the event.

### Reporting of Serious Adverse Events (SAEs)

Responsibility of investigator: The investigator must report immediately to the drug safety officer (i.e. within 24 hours of his/her becoming aware of the occurrence of the SAE) all SAEs which occur until Day 29 or until Day of Discharge, if earlier.

The preliminary information must contain at least the following data:
Identification of the reporting investigator: department name, name, institute, address, telephone number, fax, e mail, etc. Identification of the study: study code or title, respectively, investigational product(s).
Identification of the patient: assigned patient no., gender and year of birth.
Description of the SAE: symptoms/diagnosis, therapeutic measures taken, outcome as far as already known, causal relationship.

The investigator provides a complete report within a reasonable time of the initial reporting, depending on duration of the event and availability of evaluations and reports of third parties. The investigator must forward the SAE report form by fax to the responsible person for pharmacovigilance (RPPV). The original of the SAE report form remains in the patient file at the study center.

In the event of death of a study patient, which did not occur due to a COVID-19 related complication, the investigator supplies the RPPV with any additional information requested. Personal data must be made pseudonymous prior to being communicated by using the identification code of the study patient.

### Reporting of Suspected Unexpected Serious Adverse Reactions (SUSARs)

All SUSARs are reported into EudraVigilance database within a maximum of 15 days (fatal or life-threatening SUSARs within a maximum of 7 days) of first knowledge by the sponsor responsible representative (i.e. responsible person for pharmacovigilance [RPPV]) and all minimal criteria fulfilled according to chapter 5.1.6.1 of “Detailed guidance on the collection, verification and presentation of adverse reaction reports arising from clinical studies on medicinal products for human use” (ENTR/CT3). The sponsor representative also informs ethic committees and competent authorities concerned, as well as all investigators.

Relevant follow-up information of fatal or life-threatening SUSARs are communicated subsequently within an additional 8 days.

Breaking the blind of SUSARs prior to notification to European database (EudraVigilance) is done by the RPPV independent from the study team.

Any circumstances which require a review of the risk-benefit assessment of the investigational medicinal product are reported to the competent authority concerned and to the ethic committees concerned promptly in accordance with local laws and the Commission Directive 2001/20/EC.

### Final Report

After data analyses, the clinical trial results will be summarized in an integrated (statistical/ medical) report which accurately reflects the clinical data of this clinical trial. Analysis and subsequent report writing will take place once the last patient has passed Day 29. Analysis of Day 91 data will be performed once the last patient has attended Day 91 assessment.

### REFERENCES

Alam et al. Perioperative elafin for ischaemia-reperfusion injury during coronary artery bypass graft surgery: a randomised-controlled trial. Heart 2015
Alcazar et al., Elafin Treatment Rescues EGFR-Klf4 Signaling and Lung Cell Survival in Ventilated Newborn Mice. Am J Respir Cell Mol Biol 2018
Alkemade H A, de Jongh G J, Arnold W P, van de Kerkhof P C, Schalkwijk J. Levels of skin-derived antileukoproteinase (SKALP)/elafin in serum correlate with disease activity during treatment of severe psoriasis with cyclosporin A. J Invest Dermatol. 1995
Ärztezeitung 18.10.2020: Was kostet Corona das Gesundheitswesen?; https://www.aerztezeitung.de/Politik/Was-kostet-Corona-das-Gesundheitswesen-Kassendaten-geben-Einblick-413830.html
Barolet et al., Arterial elastase activity after balloon angioplasty and effects of elafin, an elastase inhibitor. Arterioscler Thromb Vasc Biol 2001
Butler M W, Robertson I, Greene C M, O'Neill S J, Taggart C C, McElvaney N G. elafin prevents lipopolysaccharide-induced AP-1 and NF-kappaB activation via an effect on the ubiquitin-proteasome pathway. J Biol Chem. (2006) 281(46):34730-5
Cowan et al., Elafin, a Serine Elastase Inhibitor, Attenuates Post-Cardiac Transplant Coronary Arteriopathy and Reduces Myocardial Necrosis in Rabbits After Heterotopic Cardiac Transplantation. Am J Respir Crit Care Med 2011
Crinnion et al., Role of neutrophil depletion and elastase inhibition in modifying skeletal muscle reperfusion injury. Cardiovasc Surg 1994
Devereux et al., Nucl. Acid Res. 12, 387-395 (1984)
European Centre for Disease Prevention and Control, Covid-19 Surveillance Report, Week 30, 2021
GMP Annex 13: https://ec.europa.eu/health/sites/default/files/files/eudralex/vol-4/2009_06_annex13.pdf
Goldman J D, Lye D C B, Hui D S, et al. Remdesivir for 5 or 10 days in patients with severe COVID-19. N Engl J Med. 2020
Guyot N, Zani M L, Maurel M C, Dallet-Choisy S, Moreau T (2005). Elafin and its precursor trappin-2 still inhibit neutrophil serine proteinases when they are covalently bound to extracellular matrix proteins by tissue transglutaminase, Biochemistry 44, 15610-8
Guyot N, Zani M L, Berger P, Dallet-Choisy S, Moreau T (2005) Proteolytic susceptibility of the serine protease inhibitor trappin-2 (pre-elafin): evidence for tryptase-mediated generation of elafin. Biol Chem. 386, 391-9
Han et al., Recombinant human elafin promotes alveologenesis in newborn mice exposed to chronic hyperoxia. Int J Biochem Cell Biol 2017
Henriksen P A et al. Adenoviral gene delivery of elafin and secretory leukocyte protease inhibitor attenuates NF-κB-dependent inflammatory responses of human endothelial cells and macrophages to atherogenic stimuli. J Immunol 2004; 172:4535-4544
Hilgendorff et al. Inhibiting lung elastase activity enables lung growth in mechanically ventilated newborn mice. Am J Respir Crit Care Med 2011
Hilgendorff et al. Neonatal mice genetically modified to express the elastase inhibitor elafin are protected against the adverse effects of mechanical ventilation on lung growth. Am J Physiol Lung Cell Mol Physiol 2012
Huang W., The Inflammatory Factors Associated with Disease Severity to Predict COVID-19 Progression. J Immunol 2021
https://www.cdc.gov/coronavirus/2019-ncov/cases-updates/cases-in-us.html
Cai Q, Chen F, Wang T, et al. Obesity and COVID-19 severity in a designated hospital in Shenzhen, China. Diabetes Care. 2020
Wu C, Chen X, Cai Y, et al. Risk factors associated with acute respiratory distress syndrome and death in patients with coronavirus disease 2019 pneumonia in Wuhan, China. JAMA Intern Med. 2020
https://www.intensivregister.de
ICH Harmonized Guideline for Good Clinical Practice E6(R1) Current Step 4 version, dated 10 Jun. 1996
Kaschwich et al. Biodistribution and pharmacokinetics of the 99mTc labeled human elastase inhibitor, elafin, in rats. Drug Metabolism and Pharmacokinetics 2016
Khan F A, Stewart I, Fabbri L, Moss S, Robinson K, Smyth A R, Jenkins G: Systematic review and meta-analysis of anakinra, sarilumab, siltuximab and tocilizumab for COVID-19. Thorax 2021
Molhuizen H O, Alkemade H A, Zeeuwen P L, de Jongh G J, Wieringa B, Schalkwijk J (1993) SKALP/elafin: an elastase inhibitor from cultured human keratinocytes. Purification, cDNA sequence, and evidence for transglutaminase cross-linking. J Biol Chem. 268(16):12028-32
Nickel et al. Elafin Reverses Pulmonary Hypertension via Caveolin-1-Dependent Bone Morphogenetic Protein Signaling. Am J Respir Crit Care Med 2015
Nickel N P, Wang L, Li G G, Spiekerkoetter E., Rabinovitch M. The elastase inhibitor elafin restores endothelial cell homeostasis In pulmonary arterial hypertension and attenuates vascular remodeling In the Sugen/hypoxia rat model. Am J Respir Crit Care Med (2013) 187:A1031
Needleman & Wunsch, J. Mol., Biol. 48,443 (1970)
NIH: Statement on Anti-SARS-CoV-2 Monoclonal Antibodies EUA, 08.07.2021
NIH: COVID-19 Treatment Guidelines (www.covid19treatmentguidelines.nih.gov)
O'Blenes et al. Gene transfer of the serine elastase inhibitor elafin protects against vein graft degeneration. Circulation 2000
Ohl M, Miller D, et al. Association of Remdesivir Treatment With Survival and Length of

Hospital Stay Among US Veterans Hospitalized With COVID-19, Jama Network Open, 2021
Ohta et al., Elafin-overexpressing mice have improved cardiac function after myocardial infarction. Am J Physiol Heart Circ Physiol 2004
Pearson & Lipman, Proc. Natl. Acad. Sci. USA 85, 2444 (1988)
Recovery Collaborative Group:
   Horby P, Lim W S, et al. Dexamethasone in hospitalized patients with COVID-19 preliminary report. N Engl J Med. 2020
   Jeronimo C M P, Farias M E L, Val F F A, et al. Methylprednisolone as adjunctive therapy for Patients hospitalized with COVID-19 (Metcovid): a randomised, double-blind, Phase IIb, placebo-controlled trial. Clin Infect Dis. 2020
RKI: Hinweise zu Erkennung, Diagnostik and Therapie von Patienten mit COVID-19, 16.07.2021
Rosa et al. In Vitro and In Vivo Models for Studying SARS-CoV-2, the Etiological Agent Responsible for COVID-19 Pandemic. Viruses 2021
Sallenave J M, Silva A (1993) Characterization and gene sequence of the precursor of elafin, an elastase-specific inhibitor in bronchial secretions. Am J Respir Cell Mol Biol. 8, 439-45
Schalkwijk J, Wiedow O, Hirose S (1999) The trappin gene family: proteins defined by an N-terminal transglutaminase substrate domain and a C-terminal four-disulphide core. Biochem J. 340, 569-77
Shavadia at al., High-throughput targeted proteomics discovery approach and spontaneous reperfusion in ST-segment elevation myocardial infarction. Am Heart J. 2020
Smith & Waterman, Adv. Appl. Math. 2, 482 (1981)
Streit V, Wiedow 0, Bartels J, Christophers E (1995) Antiprotease activity in urine of patients with inflammatory skin disorders. J Invest Dermatol. 105, 562-6
Suzuki et al., SARS-CoV-2 spike protein-mediated cell signaling in lung vascular cells. Vascular Pharmacology 2020 preprint
Tamechika I, Itakura M, Saruta Y, Furukawa M, Kato A, Tachibana S, Hirose S (1996) Accelerated evolution in inhibitor domains of porcine elafin family members. J Biol Chem. 271, 7012-8
Tiefenbacher et al., Inhibition of elastase improves myocardial function after repetitive ischaemia and myocardial infarction in the rat heart. Eur J Physiol 1997
Tsunemi M, Matsuura Y, Sakakibara S, Katsube Y (1996) Crystal structure of an elastase-specific inhibitor elafin complexed with porcine pancreatic elastase determined at 1.9 A resolution. Biochemistry 35, 11570-6
Varga, et al. Endothelial cell infection and endotheliitis in COVID-19, Lancet 2020
Wang et al. Plasma Neutrophil Elastase and Elafin as Prognostic Biomarker for Acute Respiratory Distress Syndrome: A Multicenter Survival and Longitudinal Prospective Observation Study. Shock. 2017
Wang Y, Zhang D, Du G, et al. Remdesivir in adults with severe COVID-19: a randomised, double-blind,placebo-controlled, multicentre trial. Lancet. 2020
WHO: https://cov d19.who.int/
WHO Resolution General Assembly 66/288 Health and population 141
WHO working group. A minimal common outcome measure set for COVID-19 clinical research. Lancet Infect Dis 2020
World Medical Association Declaration of Helsinki—Ethical Principles for Medical Research Involving Human Subjects, 64th WMA General Assembly, Fortaleza, Brazil, October 2013
Zaidi et al., Targeted overexpression of elafin protects mice against cardiac dysfunction and mortality following viral myocarditis. J Clin Invest 1999
Zaidi et al., Suppressed smooth muscle proliferation and inflammatory cell invasion after arterial injury in elafin-overexpressing mice. J Clin Invest 2000
Zaidi et al., Overexpression of the serine elastase inhibitor elafin protects transgenic mice from hypoxic pulmonary hypertension. Circulation 2002
Zhou F et al., Clinical course and risk factors for mortality of adult inpatients with COVID-19 in Wuhan,China: a retrospective cohort study, Lancet 2020.

All references, articles, publications, patents, patent publications, and patent applications cited herein are incorporated by reference in their entireties for all purposes. However, mention of any reference, article, publication, patent, patent publication, and patent application herein is not, and should not be, taken as acknowledgment or any form of suggestion that they constitute valid prior art or form part of the common general knowledge in any country in the world.

1. A method of treating a human patient infected with COVID-19, comprising administering to the patient a polypeptide comprising the sequence of SEQ ID NO:1, or a homologue, fragment or derivative thereof.

2. The method according to claim 1 for treating a human patient with moderate COVID-19 disease.

3. The method according to claim 1 for preventing severe COVID-19 disease in a human patient.

4. A method of treating and/or preventing disorders or diseases which are associated with SARS-CoV2 infection in a human patient, comprising administering to the patient a polypeptide comprising the sequence of SEQ ID NO:1, or a homologue, fragment or derivative thereof.

5. A method of alleviating respiratory distress associated with SARS-CoV2 infection in a human patient, comprising administering to the patient a polypeptide comprising the sequence of SEQ ID NO:1, or a homologue, fragment or derivative thereof.

6. A method of facilitating and accelerating the removal of the need for oxygen supply in a human patient infected with the SARS-CoV-2 virus, comprising administering to the patient a polypeptide comprising the sequence of SEQ ID NO:1, or a homologue, fragment or derivative thereof.

7. The method according to claim 1, wherein the patient to be treated had a positive SARS-CoV2-PCR test, is hospitalized, and is Score 4 of the WHO COVID-19 clinical progression scale (no supplemental oxygen provided).

8. The method according to claim 1, wherein the patient to be treated had a positive SARS-CoV2-PCR test, is hospitalized, and is Score 5 of the WHO COVID-19 clinical progression scale (supplemental oxygen provided by mask or nasal prongs).

9. The method according to claim 1, wherein the polypeptide comprising the sequence of SEQ ID NO:1 or homologue, fragment or derivative thereof is administered to the patient parenterally or orally.

10. The method according to claim 1, wherein the polypeptide comprising the sequence of SEQ ID NO:1 or homologue, fragment or derivative thereof is administered to the patient for a maximum of 10 days, or until the patient is without oxygen therapy for at least 24 hours.

11. The method according to claim 1, wherein the polypeptide comprising the sequence of SEQ ID NO:1 or homologue, fragment or derivative thereof is administered to the patient together with a suitable antiviral or anti-inflammatory therapy; mechanical ventilation; and/or supplemental oxygen.

12. The method according to claim 1, wherein the polypeptide comprising the sequence of SEQ ID NO:1 or homologue, fragment or derivative thereof is administered to the patient at a dosage of about 10 to about 500 mg/day.

13. The method according to claim 1, wherein the polypeptide comprising the sequence of SEQ ID NO:1 or a homologue, fragment or derivative thereof is a polypeptide having the sequence of SEQ ID NO:1 (human elafin).

14. The method according to claim 13, wherein the patient is hospitalized for COVID-19 treatment.

15. The method according to claim 13, wherein the patient to be treated had a positive SARS-CoV2-PCR test, is hospitalized, and is Score 5 of the WHO COVID-19 clinical progression scale (supplemental oxygen provided by mask or nasal prongs).

16. The method according to claim 13, wherein the patient to be treated had a positive SARS-CoV2-PCR test, is hospitalized, and is Score 5 of the WHO COVID-19 clinical progression scale (supplemental oxygen provided by mask or nasal prongs), and wherein said polypeptide is administered to the patient at a dosage of about 200 mg/day.

17. The method according to claim 13, wherein the patient to be treated had a positive SARS-CoV2-PCR test, is hospitalized, and is Score 5 of the WHO COVID-19 clinical progression scale (supplemental oxygen provided by mask or nasal prongs), and wherein said polypeptide is administered to the patient at a dosage of about 200 mg/day by intravenous infusion.

18. The method according to claim 13, wherein the patient to be treated had a positive SARS-CoV2-PCR test, is hospitalized, and is Score 5 of the WHO COVID-19 clinical progression scale (supplemental oxygen provided by mask or nasal prongs), and wherein said polypeptide is administered to the patient at a dosage of about 200 mg/day by intravenous infusion given as two infusion each comprising 100 mg of the polypeptide, where the second infusion is given 12 hours±2 hours after the first infusion.

19.-33. (canceled)

34. The method according to claim 1, wherein the polypeptide comprising the sequence of SEQ ID NO:1 or homologue, fragment or derivative thereof is administered to the patient by intravenous infusion.

35. The method according to claim 11, wherein said antiviral or anti-inflammatory therapy is selected from the group consisting of: nirmatrelvir plus ritonavir, remdesivir, monoclonal anti-SARS CoV-2 antibodies and dexamethasone.
